(19) United States
(12) Patent Application Publication
(10) Pub. No.: US 2023/0201225 A1
(43) Pub. Date: Jun. 29, 2023

(54) METHODS OF TREATING MULTIPLE SCLEROSIS

(71) Applicant: REPRISE PHARMACEUTICALS, INC., New York, NY (US)

(72) Inventor: Marc J. Simard, Baltimore, MD (US)

(21) Appl. No.: 18/118,209

(22) Filed: Mar. 7, 2023

Related U.S. Application Data
(63) Continuation of application No. 17/911,231, filed on Sep. 13, 2022.
(60) Provisional application No. 62/989,326, filed on Mar. 13, 2020.

(51) Int. Cl. A61K 31/64; A61K 31/225

(52) U.S. Cl. CPC A61K 31/64; A61K 31/225

(57) ABSTRACT

The present invention provides methods treating multiple sclerosis (MS) in a patient in need of treatment thereof. The methods comprise co-administering to the patient a fumarate or pharmaceutical acceptable salt thereof, and at least one sulfonylurea or pharmaceutical acceptable salt thereof, where the amount of the fumarate is administered at a dose lower than the therapeutically effective dose if the fumarate is administered alone.

### BACKGROUND OF THE INVENTION

Multiple sclerosis (“MS”) is an inflammatory disease of the brain and spinal cord characterized by recurrent foci of inflammation that lead to destruction of the myelin sheath. An estimated 400,000 Americans have MS, and about 2.5 million people around the world are stricken with MS. The disease generally first occurs in people between the ages of 20 and 50, although it can occur at other ages as well. The disease appears to occur twice as frequently in women as in men.

The symptoms of MS are varied, and include changes in sensation (hypoesthesia), muscle weakness, abnormal muscle spasms, or difficulty in moving; difficulties with coordination and balance (ataxia); problems in speech (dysarthria) or swallowing (dysphagia), visual problems (nystagmus, optic neuritis, or diplopia), fatigue and acute or chronic pain syndromes, bladder and bowel difficulties, cognitive impairment, or emotional symptomatology (mainly depression).

A case of MS displays one of several patterns of presentation and subsequent course. MS commonly first manifests itself as a series of attacks followed by complete or partial remissions as symptoms mysteriously lessen, only to return later after a period of stability. This type of MS is termed relapsing-remitting MS (“RRMS”). RRMS is generally characterized by unpredictable acute attacks, called “exacerbations,” with worsening of symptoms followed by full, partial, or no recovery of some function. These attacks appear to evolve over several days to weeks. Recovery from an attack takes weeks sometimes months. The disease does not worsen in the periods between the attacks. This pattern usually occurs early in the course of MS in most individuals afflicted by MS.

A second category of MS is primary-progressive MS (“PPMS”), which is characterized by a gradual clinical decline with no distinct remissions, although a patient may experience temporary plateaus or minor relief from symptoms, characterized by a gradual but steady progression of disability, without any obvious relapses and remissions. This form of disease occurs in just 15% of all people with MS, but it is the most common type of MS in people who develop the disease after the age of 40.

Another category of MS is secondary-progressive MS (“SPMS”). SPMS initially begins with a relapsing-remitting course, but later evolves into progressive disease, e.g., primary-progressive course. The progressive part of the disease may begin shortly after the onset of MS, or it may occur years or decades later. If the disease remains untreated, about 50 percent of people with RRMS will transition into secondary-progressive MS (SPMS) within a decade of the initial diagnosis.

Less commonly, patients may experience progressive-relapsing MS (“PRMS”), during which the disease takes a progressive path punctuated by acute attacks. PRMS is the least common form of the disease and is characterized by a steady progression in disability with acute attacks that may or may not be followed by some recovery. People with progressive relapsing MS initially appear to have primary progressive MS.

PRMS, SPMS, and PRMS are sometimes categorized together as types of “chronic progressive MS” or “progressive MS.” A smaller subset of patients experience “malignant MS.” Malignant MS is an aggressive and rare form of MS. It is characterized by rapidly progressive inflammation and destruction of myelin (protective covering surrounding the nerves) and increased formation of lesions and plaque in the brain and spine.

Currently, no therapy is completely effective against MS or any of the variants of MS. Furthermore, no therapy is currently approved for treatment of PPMS or SPMS. One of the major reasons for there being no approved treatment for PPMS or SPMS is an increased heterogeneity of disease presentation in SPMS patients. There is a need for improved identification and characterization of MS, including PPMS and SPMS, patient populations.

### SUMMARY OF THE INVENTION

The present invention provides methods treating multiple sclerosis (MS) in a patient in need of treatment thereof. The methods comprise co-administering to the patient a fumarate or pharmaceutical acceptable salt thereof, and at least one sulfonylurea or pharmaceutical acceptable salt thereof, where the amount of the fumarate is administered at a dose lower than the therapeutically effective dose if the fumarate is administered alone.

### BRIEF DESCRIPTION OF THE DRAWINGS

FIG. 1 depicts a comparison of the regression curves of treatment groups in mouse models. The combination of gliclazide (GCZ) and dimethyl fumarate (DMF) (bottom curve) resulted in singificatnly improved clinical scores over about a 28 day period, compared to control group, DMF treatment alone and GCZ treatment alone. There was no observed difference between the vehicle control, DMF treatment alone or GCZ treatment alone (top three curves).

### DETAILED DESCRIPTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting. It is further to be understood that the embodiments disclosed in the following subsections may be combined with other embodiments from the same or other subsections without limit.

The present invention provides methods treating multiple sclerosis (MS) in a patient in need of treatment thereof. The methods comprise co-administering to the patient a fumarate or pharmaceutical acceptable salt thereof, and at least one sulfonylurea or pharmaceutical acceptable salt thereof, where the amount of the fumarate is administered at a dose lower than the therapeutically effective dose if the fumarate is administered alone in the treatment of MS.

The subject to be treated or upon which the described methods can be applied include an adult, adolescent or child. In one specific embodiment, the subject child over 9 years of age. In another specific embodiment, the subject adolescent is less than 18 years of age. In another specific embodiment, the subject adult is 18 years of age or older. The subject can be male or female. In certain embodiments, the present invention provides an effective means for treating pregnant or lactating women who have MS. While a number of medications are currently FDA-approved for treating MS, none is specifically approved for use during pregnancy. For example, many MS patients take beta interferon (AVONEXⓇ, BETASERONⓇ and REBIFⓇ. These medicines lessen flares and slow down the spread of nerve damage and the course of MS. Beta interferon is not safe to take during pregnancy. The terms “subject” and “patient” are used interchangeably herein.

As used herein, the term Multiple Sclerosis (MS) is understood as it is in the art. Namely, MS is a chronic disease of the central nervous system marked by demyelination of neuronal axons. Although MS is often considered an autoimmune disease, other factors may also contribute to the development of MS in an individual, such as but not limited to, infections, e.g., viral infections, autoimmune disorders, environmental factors and genetic factors. Any one of these factors, or a combination thereof, may contribute to the onset of MS.

Currently, there is no single test, or even battery of tests that confirm a diagnosis of MS. Instead, current protocols for diagnosing MS involve first ruling out other possible diseases or conditions in combination with determining if the subject meets well-established clinical criteria of MS. These well-established criteria include cataloging the symptoms of the patient, reviewing the patient’s medical history, conducting a thorough neurological examination including but not limited to a magnetic resonance imaging (MRI) test, spinal fluid analysis and blood tests. After ruling out other possible causes of the symptomatic patient, a physician should find (a) evidence of damage in at least two separate areas of the central nervous system (CNS), e.g., brain, spinal cord and optic nerve, and (b) evidence that this damage occurred at least two different time points, to diagnose MS.

As used herein, the methods of treatment described herein can be applied to a patient in which a formal diagnosis of MS has not yet occurred, except that the subject should be exhibiting at least two or three symptoms of MS and the attending physician has ruled out other possible causes for these symptoms. Accordingly, a “method of treating MS,” as used herein, will include co-administration of the fumarate and sulfonylurea to a subject exhibiting at least two or three symptoms of MS and in which the attending physician has ruled out other possible causes for these symptoms. Of course, the methods of treatment described herein can also be applied to a subject in which the attending physician has documented evidence of CNS damage at two different areas within the CNS occurring at different times, such that MS has been diagnosed in the patient.

Symptoms of MS include but are not limited to headaches, blurred or double vision, red-green color distortion, pain and loss of vision, inflammation of the optic nerve, difficulty walking, paresthesia (pins and needles). Other symptoms include but are not limited to pain in the back or eyes, tremors of the hands or limbs, muscle cramps, inability to rapidly change motions, involuntary movements, muscle paralysis, muscle rigidity, muscle weakness, problems with coordination, clumsiness, muscle stiffness, muscle spasms, overactive reflexes, impaired walking or standing, partial or complete paralysis spasticity, fatigue, dizziness, heat intolerance, poor balance, vertigo, weakness, abnormality of taste, tongue numbness, difficulty swallowing, numbness of face, rapid involuntary eye movement, hearing loss, reduced sensation of touch, uncomfortable tingling and burning, excessive urination at night, leaking of urine, persistent urge to urinate, urinary retention, constipation, sleep deprivation, depression, anxiety or mood swings and speech imprediments such as slurred speech or impaired voice. Cognitive symptoms of MS include but are not limited to difficulty with concentration, attention or memory and poor judgment.

The treatment methods described herein can be used to ameliorate one or more of the aforementioned symptoms of any type of MS. As noted above, MS presents in several forms, generally characterized by three main forms: clinically isolated syndrome (CIS), non-progressive relapsing forms and progressive forms. In relapsing forms, new symptoms tend to occur in discrete attacks, but the symptoms do not necessarily tend to worsen over time. In progressive forms, however, new or previous symptoms will accumulate and or worsen over time. See, e.g., Lublin & Reingold, 1996 Neurology 46(4):907-911, which is incorporated by reference.

Accordingly, the present invention relates to methods of treatment progressive forms of MS, including primary progressive (PPMS), secondary progressive (SPMS), progressive relapsing (PRMS) and/or malignant MS (MMS). In other embodiments, the present invention relates to methods of treatment non-progressive forms of MS such as, but not limited to, relapsing-remitting MS (RRMS). For example, for primary progressive MS, three levels of diagnostic certainty are “definite,” “probable” and “possible,” based on clinical findings, abnormal cerebrospinal fluid, abnormalities on MRI of the brain and special cord and evoked potentials. According to one set of criteria, in definite PPMS, evidence of intrathecal synthesis of immunoglobulin G together with one of the following three MRI criteria is indicative of PPMS: (1) nine brain lesions, (2) two spinal cord lesions, (3) four to eight brain lesions and one spinal cord lesion. See, e.g., Thompson et al., Ann. Neurol. 47(6):831-835 (2000), which is incorporated by reference. These and other well-established diagnostic criteria can be used to diagnose an individual as having PPMS. Ultimate diagnosis will depend upon the judgment of the neurologist.

Diagnosis of individuals as having SPMS, for example, is largely based upon the judgment of the neurologist. Guidance for diagnosing MS patients can also be found in the medical literature, such as McDonald et al., Ann. Neurol., 50(1):121-127 (2001), incorporated by reference. In practice, when an individual with RRMS becomes aware of a definite deterioration in their level of function that continues for at least six months to one year which is not related to a relapse, then it is likely that the attending physician may diagnose the individual with SPMS. SPMS commonly occurs in individuals who previously manifest problems or disabilities as a result of previous MS relapses that have not recovered completely.

As noted above, the term MS includes clinically isolated syndrome (CIS). CIS is generally known as a first episode of neurological symptoms that lasts for 24 hours, with symptoms typical of MS but does not yet meet the criteria for a positive diagnosis of MS.

The term “treatment,” as used herein, means alleviation or amelioration of one or more symptoms of MS, or, in the alternative, an improvement in one or more of the physical or cognitive effects of MS. Treatment also includes amelioration or removal of all symptoms of MS that the patient is exhibiting, or, in the alternative, an improvement all of the physical or cognitive effects of MS that the subject is exhibiting.

The treatments may result in an improvement in the subject’s symptoms, and these improvements be qualitative or quantitative in nature. For example, physical disability may be measured by the subject’s Kurtzke Expanded Disability Status Scale (EDSS) score and/or wherein the accumulation of physical disability is assessed by the time to confirmed disease progression as measured by Kurtzke Expanded Disability Status Scale (EDSS) score. In certain embodiments, the treatment methods described herein result in one or more symptoms that are alleviated by at least 10%, 20%, or 30% in the Kurtzke Expanded Disability Status Scale (EDSS). The EDSS is a method of quantifying disability in multiple sclerosis. The EDSS quantifies disability in eight Functional Systems (FS) and allows neurologists to assign a Functional System Score (FSS) in each of these. The Functional Systems are: pyramidal, cerebellar, brainstem, sensory, bowel and bladder, visual & cerebral. The MS Functional Composite, which measures ambulation, hand function and speed of processing, also may be used. Visual acuity may be assessed using the Low Contrast Letter Acuity test.

Improvements in MS symptoms in the subject may also be measured using imaging techniques. One technique assesses lesion counts on MRI, adding up the number of T1-weighted gadolinium-enhancing lesions, new T2 lesions, or active, i.e., new or enlarging, T2 lesions. The evolution of active lesions into T1-hypointense lesions, so-called “black holes” may be used. Similarly, MRI-derived brain volume, which is a measure of atrophy, can also be used to measure improvements in MS symptoms. Moreover, improvements on MRI T2 lesion volume can be used alone or in combination with MRI-derived brain volume. In addition, improvements can be assessed using optical coherence tomography, which uses infrared to evaluate nerve fiber layer changes.

Other methods of assessing clinical improvements in the subject may be assessed by measuring, for example for RRMS, the annualized relapse rate, time-to-first relapse and conversion to clinically-definite MS.

The methods described herein comprise co-administering to the patient a fumarate or pharmaceutical acceptable salt thereof, and at least one sulfonylurea or pharmaceutical acceptable salt thereof, where the amount of the fumarate is administered at low doses. In one embodiment, the fumarate that is administered is dimethyl fumarate or pharmaceutical acceptable salt thereof. In another embodiment, the fumarate that is administered is diroximel fumarate or pharmaceutical acceptable salt thereof. In another embodiment, the fumarate that is administered is monomethyl fumarate or pharmaceutical acceptable salt thereof.

The term fumarate includes fumaric acid esters (FAE), fumarate esters, e.g., alkyl or dialkyl fumarate esters such as, for example, dimethyl fumarate and monomethyl fumarate. Fumarates are pharmacologically active substances used for treating hyperproliferative, inflammatory, or autoimmune disorders. Examples of fumarates include but are not limited to dimethyl fumarate (DMF), diroximel fumarate (DXF) and monomethyl fumarate (MMF). Fumarates were first used to treat psoriasis and were licensed for this indication in Germany in 1995 as FUMADERM™. (Biogen Idec, Inc., Cambridge, Mass., USA). FUMADERM™, like other fumarates, produces various undesirable side effects, including flushing, headaches, dizziness, eructation, nausea, vomiting, abdominal and intestinal cramps, and diarrhea. High concentrations of the drug released in the stomach are believed to be responsible for such side effects.

After oral intake, the main component of FUMADERM™ (dimethyl fumarate), is hydrolysed by esterases to monomethyl fumarate (MMF), the bioactive metabolite. After absorption in the small intestine, MMF is believed to interact with immunocytes in the bloodstream. The primary plasma metabolites of dimethyl fumarate are monomethyl fumarate, fumaric acid, citric acid, and glucose. Monomethyl fumarate is further metabolized in the tricarboxylic acid cycle to carbon dioxide and water.

An oral formulation of DMF was recently developed and approved for the treatment of multiple sclerosis. This formulation, TECFIDERA™ (Biogen Idec, Inc.), is available as hard gelatin delayed-release capsules containing 120 mg or 240 mg of granulated dimethyl fumarate enterically coated minitablets. TECFIDERA™ is administered at 120 mg, twice daily (240 mg/day) as an initial starting dose for seven days, followed by 240 mg twice daily (480 mg/day) as the maintenance dose. See International Patent Application Publication No. WO 2013/119677 and U.S. Pat. No. 6,509,376, which are incorporated by reference. The particular formulation of TECFIDERA™ was intended to reduce undesirable side effects associated with dimethyl fumarate by preventing release of the dimethyl fumarate in the stomach.

As used herein, the terms “initial starting dose” or “starting dose” mean a dose that is given initially, i.e., the first day that the patient ever takes or receives the drug, to the subject for a limited period of time. The starting dose is the dose at which there will a pre-conceived plan to adjust this dose, i.e., increase the dose, at a later time, after a short period of time. This period of time for this initial dosing for any of the pharmaceutically active compounds described herein can be about 2, 3, 4, 5, 6, 7, 8, 9, 10, 11, 12, 13, 14, 15, 16, 17, 18, 19, 20, 21, 22, 23, 24, 25, 26, 27, 28, 29 or about 30 days, after which an adjustment in the dosing of the active pharmaceutical will be made to achieve the maintenance dose. The period of time for this initial dosing can be longer if needed. Often times, the prescribing information in the approved pharmaceutical will provide both the starting dose as well as the length of time that the subject it to receive this starting dose.

As used herein, the term “maintenance dose” means the dose upon which the patient is administered after a pre-conceived adjustment is made to the initial starting dose. Often times, the prescribing information in the approved pharmaceutical will provide both the maintenance dose as well as the length of time that the subject it to receive the starting dose before adjusting to the maintenance does. The patient is to maintain the maintenance dose at the prescribed level with no further pre-conceived plan to adjust the dose unless and until the attending physician has data, test results or any other reason to adjust this maintenance dose, i.e., a further increase to treat the symptoms of MS. In other words, the maintenance dose is the dose at which the pharmaceutical is administered for the foreseeable future.

In some embodiments, there may be more than one “step” from the initial starting dose to the maintenance dose. These “step up” or “step down” doses “in route” to the final maintenance dose are not to be considered as the maintenance dose.

Another fumarate, VUMERITY™ (diroximel fumarate), was recently approved for treating relapsing forms of MS, clinically isolated syndrome (CIS), relapsing-remitting MS and secondary progressive disease, in adults. VUMERITY™ is available in capsules of 231 mg of DXF and is administered in doses of 231 mg, twice daily (462 mg/day) as an initial starting dose for seven days, followed by 462 mg twice daily (924 mg/day) as the maintenance dose.

Another fumarate, BAFIERTAM™ (monomethyl fumarate), is also prescribed for MS, for example RRMS. BAFIERTAM™ is delayed-release monomethyl fumarate and is available in capsules of 95 mg. The initial starting dose of BAFIERTAM™ is 190 mg (95 mg, twice per day) for seven days. The maintenance dose BAFIERTAM™ is 380 mg (190 mg, twice per day).

The methods described herein comprise co-administering to the patient a fumarate or pharmaceutical acceptable salt thereof, and at least one sulfonylurea or pharmaceutical acceptable salt thereof, where the amount of the sulfonylurea is administered at a low dose. In one embodiment, the sulfonylurea that is administered is gliclazide or pharmaceutical acceptable salt thereof. In another embodiment, the sulfonyl urea that is administered is glibenclamide or pharmaceutical acceptable salt thereof.

Sulfonylureas are anti-diabetic drugs that are not approved for treatment of MS. Sulfonylureas generally binds to the SUR1 receptor, which blocks the flow of potassium ions across the cell membrane. Examples of sulfonylureas include but are not limited to gliclazide, glipizide, glibornuride, gliquidone, glyclopyramide, glibenclamide and glimepiride. In select embodiments, the methods of treatment described herein specifically exclude administering tolbutamide or chlorpropamide as the sulfonylurea. Glibenclamide is more potent on a per-weight basis than other sulfonylureas and so, in general, unless otherwise indicated, dosing conversions from glibenclamide to the other inhibitors may be made by multiplying the dosing information for glibenclamide according to the following:

| Sulfonylurea | Multiply Glibenclamide Dosing by About: |
|---|---|
| Glibornuride | 4.2 |
| Gliclazide | 13.3 |
| Gliquidone | 5 |
| Glyclopyramide | 42 |

Glibenclamide, also known as glyburide, is marketed in the United States as DIABETA™, GLYNASE™ and GLYNASE PRESTAB™ and is available in tablet form. Glibenclamide, DIABETA™, is formulated in 1.25 mg, 2.5 mg, and 5 mg tablet strengths. While there is no set dosing regimen for using glibenclamide, DIABETA™, to manage diabetes, a typical starting dose is often 1.25 mg to 5 mg daily, with the maintenance dose often settling in with a range of about 1.25 mg to 20 mg daily, with maximum doses of more than 20 mg daily are not recommended. Glibenclamide, GLYNASE PRESTAB™, is formulated in in 1.5 mg, 3 mg, and 6 mg tablet strengths. While there is no set dosing regimen for using glibenclamide, GLYNASE PRESTAB ™, to manage diabetes, a typical starting dose is often 1.5 mg to 3 mg daily, with the maintenance dose often settling in with a range of about 0.75 mg to 12 mg daily.

Specific combinations of administering at least one active fumarate and at least one active sulfonylurea in the methods of treatment are noted in the chart below.

| | | |
|---|---|---|
| dimethyl fumarate (DMF) | | diroximel fumarate (DXF) |
| Gliclazide (GCZ) | X | X |
| Glipizide (GPZ) | X | X |
| Glibornuride (GBN) | X | X |
| Gliquidone (GQD) | X | X |
| Glyclopyramide (GPA) | X | X |
| Glibenclamide (GBC) | X | X |
| Glimepiride (GMP) | X | X |

In select embodiments of the invention, the methods comprise administering the fumarate at maintenance doses lower than would be prescribed for treating MS, if the fumarate were administered alone. In additional select embodiments of the invention, the methods comprise administering the sulfonylurea at maintenance doses lower than would be prescribed for treating diabetes, if the sulfonylurea were administered alone. In more select embodiments of the invention, the methods comprise administering the fumarate at maintenance doses lower than would be prescribed for treating MS and administering the sulfonylurea at maintenance doses lower than would be prescribed for treating diabetes, if each of the fumarate and sulfonylurea were administered alone.

In certain embodiments, the total daily maintenance dose of DMF will be less than 480 mg/day. In one dosing regimen, the DMF is administered in a dose of equal to or less than 240 mg per day for about 5 - 10 days as the initial starter dose, before the dose is increased to less than 480 mg/day (the maintenance dose). In select embodiments, the maintenance dose of DMF is between about 50-350 mg per day (total), with the DMF being administered one, two, three or four times per day to reach the total daily maintenance dose. In certain embodiments, in one dosing regimen, the DMF is administered in a dose of about 120 mg per day for about 5 - 10 days as the initial starter dose, before the dose is increased to about 240 mg/day (the maintenance dose). In specific select embodiments, the maintenance dose of DMF is about 120 mg/day or 240 mg per day (total), with the DMF being administered one, two, three or four times per day to reach the total daily maintenance dose. In even more select embodiments, the DMF is administered without an initial starting dose, i.e., the initial starting dose and the maintenance dose of the DMF are the same such that there is no pre-conceived plan to adjust the dose of DMF after the subject begins taking or receiving the DMF.

In certain embodiments, the total daily maintenance dose of DXF, will be less than 924 mg/day. In one dosing regimen, the DXF is administered in a dose of equal to or less than 462 mg per day for about 5 - 10 days as the initial starter dose, before the dose is increased to less than 962 mg/day (the maintenance dose). In select embodiments, the maintenance dose of DXF is between about 100-750 mg per day (total), although it may be as low as 85 mg per day (total), with the DXF being administered one, two, three or four times per day to reach the total daily maintenance dose. In one dosing regimen, the DXF is administered in a dose of about 231 mg per day for about 5 - 10 days as the initial starter dose, before the dose is increased to about 462 mg/day (the maintenance dose). In specific select embodiments, the maintenance dose of DXF is about 231 mg/day or 462 mg per day (total), with the DXF being administered one, two, three or four times per day to reach the total daily maintenance dose. In even more select embodiments, the DXF is administered without an initial starting dose, i.e., the initial starting dose and the maintenance dose of the DXF are the same such that there is no pre-conceived plan to adjust the dose of DXF after the subject begins taking or receiving the DXF.

In certain embodiments, the total daily maintenance dose of MMF, will be less than 380 mg/day. In one dosing regimen, the MMF is administered in a dose of equal to or less than 190 mg per day for about 5 - 10 days as the initial starter dose, before the dose is increased to less than 380 mg/day (the maintenance dose). In select embodiments, the maintenance dose of MMF is between about 50-300 mg per day (total), with the MMF being administered one, two, three or four times per day to reach the total daily maintenance dose. In one dosing regimen, the MMF is administered in a dose of about 95 mg per day for about 5 - 10 days as the initial starter dose, before the dose is increased to about 190 mg/day (the maintenance dose). In specific select embodiments, the maintenance dose of MMF is about 95 mg/day or 190 mg per day (total), with the MMF being administered one, two, three or four times per day to reach the total daily maintenance dose. In even more select embodiments, the MMF is administered without an initial starting dose, i.e., the initial starting dose and the maintenance dose of the MMF are the same such that there is no pre-conceived plan to adjust the dose of MMF after the subject begins taking or receiving the MMF.

In another embodiment, the methods comprise administering a sulfonylurea and a fumarate at a maintenance dose provided in one or more dosage forms, wherein the subject administered the dose form(s) exhibit one or more pharmacokinetic parameters comprising: (a) a mean plasma monomethyl fumarate Cₘₐₓ ranging from about 0.4 mg/L to about 2.41 mg/L, or (b) a mean plasma monomethyl fumarate AUCₒᵥₑᵣₐₗₗ ranging from about 3.2 h*mg/L to about 11.2 h*mg/L.

In another embodiment, the methods comprise administering a sulfonylurea and a fumarate at a maintenance dose provided in one or more dosage forms, wherein the subject administered the dose form(s) twice-daily exhibit one or more pharmacokinetic parameters comprising: (a) a mean plasma monomethyl fumarate Cₘₐₓ ranging from about 1.0 mg/L to about 3.4 mg/L, or (b) a mean plasma monomethyl fumarate AUCₒᵥₑᵣₐₗₗ ranging from about 4.81 h*mg/L to about 11.2 h*mg/L.

In another embodiment, the methods comprise administering a sulfonylurea and a fumarate at a maintenance dose provided in one or more dosage forms, wherein the subject administered the dose form(s) exhibits one or more pharmacokinetic parameters comprising: (a) a mean plasma monomethyl fumarate Cₘₐₓ ranging from about 0.4 mg/L to about 2.41 mg/L, (b) a mean plasma monomethyl fumarate AUC_{0→12h} ranging from about 0.5 h*mg/L to about 2.5 h*mg/L, or (c) a mean AUC_{0→∞} ranging from about 0.5 h*mg/L to about 2.6 h*mg/L.

In another embodiment, the methods comprise administering a sulfonylurea and a fumarate at a maintenance dose provided in one or more dosage forms, wherein the subject administered the dose form(s) twice daily exhibit one or more pharmacokinetic parameters comprising: (a) a mean plasma monomethyl fumarate Cₘₐₓ ranging from about 1.0 mg/L to about 3.4 mg/L, (b) a mean plasma monomethyl fumarate AUC_{0→12h} ranging from about 1.0 h*mg/L to about 5.5 h*mg/L, or (c) a mean AUC_{0→∞} ranging from about 1.0 h*mg/L to about 5.6 h*mg/L.

In select embodiments of the invention, the methods comprise administering the fumarate to the human subject at maintenance doses lower than would be prescribed for treating MS, if the fumarate were administered alone. In additional select embodiments of the invention, the methods comprise administering the sulfonylurea to the human subject at maintenance doses lower than would be prescribed for treating diabetes, if the sulfonylurea were administered alone. In more select embodiments of the invention, the methods comprise administering the fumarate at maintenance doses lower than would be prescribed for treating MS and administering the sulfonylurea at maintenance doses lower than would be prescribed for treating diabetes, if each of the fumarate and sulfonylurea were administered alone.

In particular, the methods comprise administering the sulfonylurea at low doses. In one specific embodiment, GCZ is administered to the human subject at a maintenance dose of equal to or less than about 60 mg/day. In one specific embodiment, GCZ is administered at a maintenance dose of equal to or less than about 60 mg/day, with the GCZ being administered one, two, three or four times per day to reach the total daily maintenance dose. In one specific embodiment, GCZ is administered at a maintenance dose of between about 1 mg per day to about 60 mg/day, 2 mg per day to about 55 mg/day, 3 mg per day to about 50 mg/day, 4 mg per day to about 45 mg/day, 5 mg per day to about 40 mg/day, or about 6 mg per day to about 35 mg per day, with the GCZ being administered one, two, three or four times per day to reach the total daily maintenance dose.

In one specific embodiment, GBC is administered to the human subject at a maintenance dose of equal to or less than about 5 mg/day. In one specific embodiment, GBC is administered at a maintenance dose of equal to or less than about 5 mg/day, with the GBC being administered one, two, three or four times per day to reach the total daily maintenance dose. In one specific embodiment, GBC is administered at a maintenance dose of between about 0.2 mg per day to about 2.5 mg per day, 0.3 mg per day to about 2.0 mg per day, 0.4 mg per day to about 1.5 mg per day, 0.5 mg per day to about 1.0 mg per day, with the GBC being administered one, two, three or four times per day to reach the total daily maintenance dose.

In one specific embodiment, GPZ is administered to the human subject at a maintenance dose of equal to or less than about 5 mg/day. In one specific embodiment, GPZ is administered at a maintenance dose of equal to or less than about 3.5 mg/day, with the GPZ being administered one, two, three or four times per day to reach the total daily maintenance dose. In one specific embodiment, GPZ is administered at a maintenance dose of between about 0.2 mg per day to about 5.0 mg per day, 0.3 mg per day to about 4.5 mg per day, 0.4 mg per day to about 4.0 mg per day, 0.5 mg per day to about 3.5 mg per day, with the GPZ being administered one, two, three or four times per day to reach the total daily maintenance dose.

In one specific embodiment, GBN is administered to the human subject at a maintenance dose of equal to or less than about 12.5 mg/day. In one specific embodiment, GBN is administered at a maintenance dose of equal to or less than about 8 mg/day, with the GBN being administered one, two, three or four times per day to reach the total daily maintenance dose. In one specific embodiment, GBN is administered at a maintenance dose of between about 0.2 mg per day to about 12.5 mg per day, 0.5 mg per day to about 12.0 mg per day, 1.0 mg per day to about 11.5 mg per day, 1.5 mg per day to about 11.0 mg per day, 2.0 mg per day to about 10.5 mg per day, 2.5 mg per day to about 10.0 mg per day, 3.0 mg per day to about 9.5 mg per day, 3.5 mg per day to about 9.0 mg per day, 4.0 mg per day to about 8.5 mg per day, 4.0 mg per day to about 8.0 mg per day, with the GBN being administered one, two, three or four times per day to reach the total daily maintenance dose.

In one specific embodiment, GQD is administered to the human subject at a maintenance dose of equal to or less than about 40 mg/day. In one specific embodiment, GQD is administered at a maintenance dose of equal to or less than about 30 mg/day, with the GQD being administered one, two, three or four times per day to reach the total daily maintenance dose. In one specific embodiment, GQD is administered at a maintenance dose of between about 1 mg per day to about 40 mg per day, 5 mg per day to about 38 mg per day, 10 mg per day to about 36 mg per day, 15 mg per day to about 34 mg per day, 20 mg per day to about 32 mg per day and 25 mg per day to about 30 mg per day, with the GQD being administered one, two, three or four times per day to reach the total daily maintenance dose.

In one specific embodiment, GPA is administered to the human subject at a maintenance dose of equal to or less than about 125 mg/day. In one specific embodiment, GPA is administered at a maintenance dose of equal to or less than about 100 mg/day, with the GPA being administered one, two, three or four times per day to reach the total daily maintenance dose. In one specific embodiment, GPA is administered at a maintenance dose of between about 10 mg per day to about 125 mg per day, 15 mg per day to about 120 mg per day, 20 mg per day to about 115 mg per day, 25 mg per day to about 110 mg per day, 30 mg per day to about 105 mg per day, 35 mg per day to about 100 mg per day, 40 mg per day to about 95 mg per day, 45 mg per day to about 90 mg per day, 50 mg per day to about 85 mg per day and 55 mg per day to about 80 mg per day, with the GPA being administered one, two, three or four times per day to reach the total daily maintenance dose.

In one specific embodiment, GMP is administered to the human subject at a maintenance dose of equal to or less than about 2 mg/day. In one specific embodiment, GMP is administered at a maintenance dose of equal to or less than about 1 mg/day, with the GMP being administered one, two, three or four times per day to reach the total daily maintenance dose. In one specific embodiment, GMP is administered at a maintenance dose of between about 0.1 mg per day to about 2.0 mg per day, 0.2 mg per day to about 1.8 mg per day, 0.3 mg per day to about 1.6 mg per day, 0.4 mg per day to about 1.4 mg per day, 0.5 mg per day to about 1.2 mg per day and 0.6 mg per day to about 1.0 mg per day, with the GMP being administered one, two, three or four times per day to reach the total daily maintenance dose.

Optionally, the effective dose level is one that reaches a maximum sulfonylurea plasma concentration level (denoted as “Cₘₐₓ”). For glibenclamide a target Cₘₐₓ is generally about 1 ng/mL to about 30 ng/mL when administered with dimethyl fumarate. Suitable maximum sulfonylurea include about 30 ng/mL, about 28 ng/mL, about 26 ng/mL, about 24 ng/ML, about 22 ng/mL, about 20 ng/mL, about 18 ng/mL, about 16 ng/mL, about 14 ng/mL, about 12 ng/mL, about 10 ng/mL, about 8 ng/mL, about 6 ng/mL, about 4 ng/mL, about 2 ng/mL, or about 1 ng/mL, or similar concentration levels. A suitable maximum concentration level may also fall in the range of about 1-2 ng/mL, about 2-4 ng/mL, about 4-6 ng/mL, about 6-8 ng/mL, about 8-10 ng/mL, about 10-12 ng/mL, about 12-14 ng/mL, about 14-16 ng/mL, about 16-18 ng/mL, about 18-20 ng/mL, about 20-22 ng/mL, about 22-24 ng/mL, about 24-26 ng/mL, about 26-28 ng/mL, or about 28-30 ng/mL. It is understood that any dosage levels substantially similar to those listed are covered by the present invention. With reference to the foregoing with respect to glibenclamide, the ranges can be multiplied by the following factors in order to obtain the analogous ranges for the following other sulfonylureas: glibornuride (4.2); gliclazide (13.3); gliquidone (5); and glyclopyramide (42). The ranges are intended to encompass analogous ranges measured in any units of weight of drug per any unit of volume. Suitable maximum concentrations of the second therapeutically active agent are in the same ranges as that of sulfonylureas. The maximum concentration of the sulfonylurea and the fumarate may be substantially the same, or may vary

Optionally, the effective dose level is one that achieves a steady-state sulfonylurea concentration. For example, for glibenclamide, this would range from about 3.0 ng/mL to about 30.0 ng/mL when administered with a fumarate. Thus, in embodiments, treatment will result in stead-state blood plasma concentrations of about 30 ng/mL, about 27 ng/mL, about 24 ng/mL, about 21 ng/mL, about 18 ng/mL, about 15 ng/mL, about 12 ng/mL, about 9 ng/mL, about 6 ng/mL, about 3 ng/mL, or anywhere between the listed concentrations. In other embodiments, the desired effective steady-state concentration may be about 3.0-5.0 ng/mL, or about 5.0-7.0 ng/mL, or about 7.0-10.0 ng/mL, or about 10.0-12.0 ng/mL, or about 12.0-14.0 ng/mL, or about 14.0-16.0 ng/mL, or about 16.0-18.0 ng/mL, or about 18.0-20.0 ng/mL, or about 20.0-22.0 ng/mL, 22.0-24.0 ng/mL, or about 24.0-26.0 ng/mL, or about 26.0-28.0 ng/mL, or about 28.0-30.0 ng/mL, or combinations thereof. In further embodiments, a steady-state concentration of about 3.0 ng/mL to about 30.0 ng/mL, or about 5.0 ng/mL to about 28.0 ng/mL, or about 7.0 ng/mL to about 26.0 ng/mL, or about 9.0 ng/mL to about 24.0 ng/mL, or about 11.0 ng/mL to about 22.0 ng/mL, or about 13.0 ng/mL to about 20.0 ng/mL, or about 15.0 ng/mL to about 18.0 ng/mL, or about 16.0 ng/mL to about 17.0 ng/mL, or combinations thereof may be desired. With reference to the foregoing with respect to glibenclamide, the ranges can be multiplied by the following factors to obtain the analogous ranges for the following other sulfonylureas: glibornuride (4.2), gliclazide (13.3), gliquidone (5) and glyclopyramide (42). The desired steady-state concentration may vary depending on several factors, including the likelihood and/or severity of MS, and may change over time. The ranges disclosed are exemplary and are intended to encompass analogous ranges measured in any units of weight per volume. The steady-state concentration of the second therapeutically active agent may be in the same ranges as that of sulfonylureas. The steady state concentrations of the sulfonylurea and the fumarate may be substantially the same, or may vary.

The specific effective dose of the fumarate and/or the sulfonylurea for any particular patient will depend on a variety of factors including the severity or likelihood of the multiple sclerosis, activity of the specific compound employed, the age, body weight, general health, sex and diet of the patient, the preparation of the specific compound, the time and route of administration, the duration of administration, therapeutic agents used in combination or coinciding with the specific sulfonylurea employed, and like factors known in the medical arts. The selected dose of the sulfonylurea and/or fumarate may also change over time as the MS symptoms worsen or improve. The number of and frequency of co-administrations of the sulfonylurea with the fumarate may vary depending upon the likelihood or severity of the MS symptoms and the patient specific response to the particular sulfonylurea administered with the fumarate.

The sulfonylurea and fumarate may be administered concurrently or sequentially in any order. In one embodiment, the sulfonylurea may be administered before the fumarate, or, in another embodiment, the sulfonylurea may be administered after the fumarate. The manner and duration of administering the sulfonylurea and fumarate may vary. In certain embodiments, the first amount of the sulfonylurea and the first amount of the fumarate thereof are administered concurrently. Alternatively, the sulfonylurea and the fumarate can be administered sequentially. For instance, in one dosing regimen, the patient will receive a first administration of the sulfonylurea, then receive a first administration of the fumarate, then receive a second administration of the sulfonylurea, and then receive a second administration of the fumarate, with the cycle repeating accordingly.

The fumarate and the sulfonylurea at the doses described above, can be administered in separate unit dosages or as single unit dosage forms. In such embodiments, the fumarate can be formulated in a variety of formulations, such as tablets, capsules, gel capsules, or other know oral delivery systems. The composition comprising the fumarate may be formulated as a controlled-release or extended-release formulation to minimize known adverse effects associated with fumarates. In another aspect, the fumarate is encapsulated in a soft capsule.

As one example, the pharmaceutical composition may comprise about 80 mg to about 110 mg of a fumarate in an immediate releasing single-phase non-aqueous liquid vehicle, as described in U.S. Pat. No. 9,820,961, which is hereby incorporated by reference in its entirety.

The fumarate may be used as an oral preparation in the form of tablets, micro-tablets, pellets or granulates, optionally in capsules or sachets. Preparations in the form of micro-tablets or pellets, optionally filled in capsules or sachets are envisioned and are also a subject matter of the invention. The oral preparations may be provided with an enteric coating. Capsules may be soft or hard gelatin capsules. Other suitable examples include those disclosed in U.S. Pat. No. 7,320,999.

In other embodiments, the fumarate will be administered less frequently than the sulfonylurea. The frequency for the fumarate administration will vary depending on the particular sulfonylurea administered in combination. By way of example, in one embodiment, a patient will receive a daily administration of the sulfonylurea and will receive an administration of fumarate once per two days, once per three days, once per four days, once per week, or once per two weeks. The combination of the sulfonylurea with the fumarate permits, in certain embodiments, a decreased frequency of administration of the fumarate without substantial decrease in the efficacy of the treatment.

In other embodiments, the co-administration of the sulfonylurea and the fumarate will generally occur for an extended period of time, such as a period of about several days, or about 1 week, or about 2 weeks, or about 3 weeks, or more until some relief of symptoms is experienced, or indefinitely to prevent or reduce the likelihood of symptoms from reoccurring.

In some embodiments, administration of the sulfonylurea and/or the fumarate is achieved by injection. An injection is an intravenous administration that may be continuous or bolus in form. A bolus injection refers to administration of the sulfonylurea and/or the fumarate in a single injection that lasts for a relatively short period of time, usually a period of about 3 minutes or less. Several bolus injections may be administered in series for any of the durations disclosed above.

In some embodiments, the sulfonylurea and the fumarate are administered orally. The oral administration may be via capsules, tablets, pills, powders, liquid suspension, or other commonly used oral administration forms. Oral administration may occur prior to, during, or after the onset of the MS symptoms, or any combination thereof. In further embodiments, the oral administration may be combined with an injection, infusion, nasal, or transdermal administration route disclosed herein or combinations thereof. The oral administration may be accomplished in a manner such that the fumarate is contained in an extended-release or controlled-release formulation.

In some co-administration embodiments, the sulfonylurea and the fumarate are administered at substantially the same time. In other co-administration embodiments, one of the sulfonylurea or the fumarate are wholly administered prior to the other of the sulfonylurea and the fumarate. The time period between the administration of the sulfonylurea and the administration of the fumarate and/or the time period between administration of the fumarate and the administration of the sulfonylurea may be of any of the duration, provided that the physiological or therapeutic effects of the sulfonylurea and the fumarate overlap. In some embodiments, the administration of either the sulfonylurea or the fumarate can be initiated first and the other can be initiated second, and the period before initiation of administration of the first and initiation of administration of the second may be any time period such that there exists a period wherein both the sulfonylurea and the fumarate are simultaneously administered. The duration of the simultaneous administration may be the entire length of one, both, or neither of the sulfonylurea administration and the fumarate administration.

This claimed combination in the methods described herein provides unexpected efficacy in treating MS, even though the fumarate being administered is below currently known therapeutically effective doses. In the alternative, another embodiment of the present invention is directed to a method of increasing the effectiveness of a fumarate when administered to treat a patient who has multiple sclerosis. In these embodiments, the increased effectiveness is observed in patients with primary progressive MS, secondary progressive MS, progressive-relapsing MS, malignant MS or non-progressive relapsing-remitting MS.

In one specific embodiment, the co-administration methods of the present invention may be used to treat, retard the progression of, delay the onset of, prevent or reduce the likelihood of, amelioration of, or reduce the symptoms of a MS in the human patient. For example, the co-administration methods of the present invention may be result in reduction or alleviation of one or more symptoms of MS selected from the group consisting of MRI-monitored multiple sclerosis disease activity, relapse rate, accumulation of physical disability, frequency of relapse, frequency of clinical exacerbation, brain atrophy, risk of confirmed progression and time to confirmed disease progression. In one specific example, the co-administration methods of the present invention may be used to reduce accumulation of physical disability as measured by the subject’s Kurtzke Expanded Disability Status Scale (EDSS) score.

In another embodiment, the co-administration methods of the present invention may be used to reduce the number, reduce the severity, delay the onset of or eliminate the occurrence of side effects associated with administration of fumarate for MS. For example, the methods of the present invention may result in a reduction of less than about 0.224 annualized relapse rate relative to baseline in the subject without substantially inducing one or more of flushing, abdominal pain, diarrhea or nausea in the subject. The baseline is determined according the standard diagnostic tests for MS patients, and example of which is available on the world wide web at www.nationalmssociety.org/For-Professionals/Clinical-Care/Managing-MS/Rehabilitation/Rehabilitation-Paradigm/Baseline-Evaluation. In one aspect, the subject experiences a reduction of one or more of flushing, abdominal pain, diarrhea, and nausea compared to a subject not practicing the co-administration methods of the present invention, i.e., compared to a subject taking or receiving only a fumarate at the prescribed levels, which are higher than those disclosed and claimed herein.

As used herein, the term “effective amount” or “effective dose” refers to the amount of an active agent sufficient to provide a therapeutic of physiologically desired effect. An effective amount can be administered in one or more administrations, applications or dosages and is not intended to be limited to a particular formulation or administration route.

The foregoing description of embodiments has been presented for purposes of illustration and description only. It is not intended to be exhaustive or to limit the application to the precise form disclosed, and modifications and variations are possible and/or would be apparent in light of the above teachings or may be acquired from practice of the application. The embodiments were chosen and described in order to explain the principles of the application and its practical application to enable one skilled in the art to utilize the application in various embodiments and with various modifications as are suited to the particular use contemplated. It is intended that the scope of the application be defined by the claims appended hereto and that the claims encompass all embodiments of the application, including the disclosed embodiments and their equivalents.

### EXAMPLES

Embodiments will be further described with reference to the following Examples, which are provided for illustrative purposes only and should not be used to limit the scope of or construe the invention.

The rodent EAE model is the most widely used model of MS, but it suffers from the deficiency of not following the natural history found in the human disease. As described herein, most MS patients undergo a period of relapsing-remitting disease, which ultimately converts to a progressive disease. The EAE model displays neither a relapsing-remitting phase, nor does it enter a progressive phase. Notwithstanding this, the model has been used to successfully screen candidate molecules that have been ultimately approved for use in humans to treat RRMS. The deficiency of this model is significant because none of the agents identified using the EAE has undergone successful development for progressive forms of MS.

It has recently been described that when the EAE model is applied to non-obese diabetic (NOD) mice, the natural history closely mimics the human progression from relapsing-remitting to progressive disease. Unlike the situation in wild-type mice, the NOD mice undergo a period of relapsing-remitting through about post-induction day 80-90 and then the disease become progressive.

Another deficiency with the prior testing is that most models in the literature employ a prophylactic approach, where drug treatment is begun prior to the onset of symptoms (e.g., hindlimb dysfunction/paralysis). This is unrealistic in that human MS patients are never treated before the disease presents clinically. The following examples, therefore, are based on a model that precisely recapitulates the natural history of human MS and they employ a realistic treatment regimen that begins only after the disease is detected clinically (i.e., it is true treatment and not prophylaxis).

Methods and formulas for determining equivalent dosing between animals and humans are well-known. For example, Nair, A., J. Basic Clin. Pharma., 7:27-31 (2016), which is incorporated by reference, discloses dose conversion calculations for converting animal doses to human doses based on the animals weight and body surface area, among other factors. One of skill in the art will readily be able to convert the animal doses provided herein to a human equivalent dose (HED) by dividing the mouse dose, on a mg/kg basis by 12.3 (assuming a 20 g mouse with a body surface area of 0.007 m²).

### Example 1 - Enhanced Treatment Effects of Low Dose DMF in an NOD EAE Relapsing-Remitting Model in the Presence of Low Dose Glibenclamide

Experimental allergic encephalitis (EAE) is induced in female C57BL/6 mice (10 weeks old) by subcutaneous injection in the flank regions (left and right sides) with 200 µl total of an emulsion of MOG₃₅₋₅₅ peptide (200 µg in 100 µl phosphate buffered saline plus 100 µl of complete Freund’s adjuvant containing 200 ng of heat-inactivated Mycobacterium tuberculosis). Each mouse then receives 200 ng of pertussis toxin intraperitoneally (IP) on the day of immunization (day 0) and then again 48 h later (day 2). Neurological assessments are reported using a five-point standardized rating scale to evaluate motor deficit: 0, no deficit; 1, tail paralysis; 2, unilateral hind limb weakness; 3, incomplete bilateral hind limb paralysis and/or partial forelimb weakness; 4, complete hind limb paralysis and partial forelimb weakness; 5, moribund state or death.

At day 18 - 21 post-immunization, the mice with a score 2 or more are distributed over the different groups to start the treatment. Animals reaching score 2 are divided into 5 groups (7-8 per group): vehicle control; glibenclamide 0.25 mg/kg (0.125 mg/kg BID); glibenclamide 0.5 mg/kg (0.25 mg/kg BID); DMF 30 mg/kg (15 mg/kg BID); and glibenclamide 0.25 mg/kg plus DMF 30 mg/kg (0.125 mg/kg BID and 15 mg/kg BID, respectively). Animals are treated in the morning and the afternoon according to their treatment assignment for 28 days. Neurological assessments are done daily to monitor disease progression

The DMF is prepared as a suspension (15 mg/ml) in 0.8 % hydroxypropyl methylcellulose (HPMC) weekly and kept at 4° C. under constant stirring. DMF is diluted into 200 µl HPMC, corresponding to a dose of 15 mg/kg body weight, and is administered twice daily (30 mg/kg/day) by oral gavage through a bulb-tipped curved gastric gavage needle by trained operators.

A stock solution of glibenclamide (15 mg/ml) is prepared by placing glibenclamide (#G2539; meets USP testing; Sigma-Aldrich) into dimethyl sulfoxide (DMSO). Prior to treatment, the stock solution is diluted into phosphate buffered saline (PBS). Mice are treated with 200 µL of the appropriate dilution intraperitoneally twice per day for a total daily dose of 0.25 mg/kg or 0.50 mg/kg daily for the low and high dose, respectively.

TABLE 1

Study Design in C57B/6 Mice

| Group No. | EAE Induction | Treatment | Dose Level (mg/kg) | Treatment Regimen |
|---|---|---|---|---|
| 1 | MOG + CFA via SC on Day 0 and two injections of PTx via IP on Days 0 and 2 | DMF | 15 BID po | Daily dosing BID upon onset of disease (~Day 18) |
| 2 | | GBC Low | 0.125 BID ip | |
| 3 | | GBC High | 0.25 BID ip | |
| 4 | | DMF + GBC Low | 15 BID po + 0.125 BID ip | |
| 5 | | Vehicle | NA | |

### Example 2 - Enhanced Treatment Effects of Low Dose Dimethyl Fumarate in an NOD Relapsing-Remitting EAE Model in the Presence of Low Dose Gliclazide

Experimental allergic encephalitis (EAE) was induced in female C57BL/6 mice as described in Example 1. Animals reaching clinical score of 2 were divided into 4 groups (9-12 per group): vehicle control; gliclazide (GCZ) 10 mg/kg (5 mg/kg BID); DMF 30 mg/kg (15 mg/kg BID); and gliclazide 10 mg/kg plus DMF 30 mg/kg (5 mg/kg BID and 15 mg/kg BID, respectively). Animals were treated in the morning and the afternoon according to their treatment assignment for 28 days. Neurological assessments were done daily to monitor disease progression.

A gliclazide stock suspension was prepared at 7.5 mg/ml (G2167 SIGMA) weekly in 0.8% sodium carboxymethylcellulose (SCMC) vehicle (419273 SIGMA). The stock suspension was kept at 4° C. under constant stirring and diluted daily in 200 µl of vehicle at the concentration desired to administer via oral gavage. DMF was prepared as it was in Example 1.

TABLE 2

Study Design in C57B/6 Mice

| Group No. | EAE Induction | Treatment | Dose Level (mg/kg) | Treatment Regimen |
|---|---|---|---|---|
| 1 | MOG + CFA via SC on Day 0 and two injections of PTx via IP on Days 0 and 2 | DMF | 15 BID po | Daily dosing BID upon onset of disease (~Day 18) |
| 2 | | GCZ | 5 BID ip | |
| 3 | | DMF+GCZ | 15 BID po; 5 BID ip | |
| 4 | | Vehicle | N/A | |

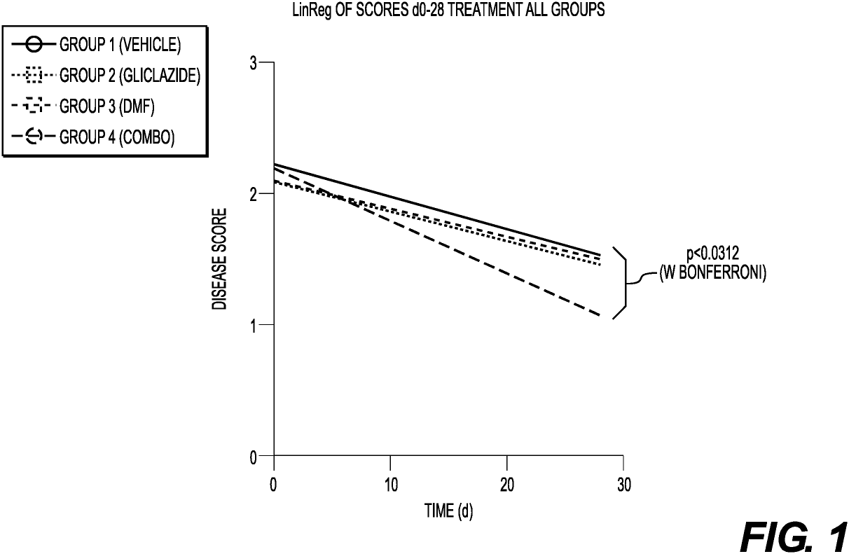

Results are shown in FIG. 1. The curve for each group was fitted to a line and then statistical comparisons were made. Specifically, the combination of gliclazide and DMF (bottom curve) resulted in singificatnly improved clinical scores over about a 28 day period, compared to control group, DMF treatment alone and GCZ treatment alone. There was no observed difference between the vehicle control, DMF treatment alone or GCZ treatment alone (top three curves).

### Example 3 - Enhanced Treatment Effects of Low Dose Dimethyl Fumarate in an NOD Relapsing-Remitting EAE Model in the Presence of Low Dose Glibenclamide

To induce active MOG₃₅₋₅₅-EAE, 10 week-old female NOD/ShiLtJ mice are immunized with MOG₃₅₋₅₅ (200 µg/injection) and M. tuberculosis (TB; 200 µg/injection) in M. butyricum-containing complete Freund’s adjuvant (CFA), as well as pertussis toxin (PTx; 500 ng/injection). On post-induction day (pid)-0, mice receive two MOG₃₅₋₅₅ emulsion injections [subcutaneous (s.c.); 0.05 mL/injection], one near the left axillary lymph nodes and one near the left inguinal lymph nodes. PTx is also administered (intraperitoneal (IP); 0.30 mL/injection). On pid-2, mice receive another PTx injection. On pid-7, mice receive two MOG₃₅₋₅₅ emulsion injections (s.c.; 0.05 mL/injection), this time near the right axillary and inguinal lymph nodes.1 Treatment of animals with DMF and or glibenclamide, as described below, commences after the onset and maximal appearance of clinical symptoms, at about pid-18 and continued through pid-40.

Dimethyl fumarate is prepared as above. A dose of 200 µl, corresponding to a dose of 15 mg/kg body weight (for a mouse weighing about 33 g) is administered twice daily (30 mg/kg/day) by oral gavage through a bulb-tipped curved gastric gavage needle by trained operators. Glibenclamide is prepared as above and diluted to the appropriate concentrations, e.g., 25 µL of stock solution diluted into 9.975 mL of PBS for the low dose and 50 µL into 9.95 mL for the high dose. Mice are treated with 200 uL of the appropriate dilution intraperitoneally twice per day for a total daily dose of 2.5 ug and 5 ug daily for the low and high dose, respectively. For a 33 g mouse, this equates to a dose of about 0.075 and 0.15 mg/kg/day.

TABLE 3

Study Design in NOD/ShiLtJ mice

| Group No. | EAE Induction | Treatment | Dose Level (mg/kg) | Treatment Regimen |
|---|---|---|---|---|
| 1 | MOG + CFA via SC on Days 1 and 8 and two injections of PTx via IP on Days 1 and 2 | DMF | 15 BID po | Daily dosing BID upon onset of disease (~Day 10-12) |
| 2 | | GBC Low | 0.038 BID ip | |
| 3 | | GBC High | 0.075 BID ip | |
| 4 | | DMF + GBC Low | 15 BID po + 0.038 BID ip | |
| 5 | | DMF + GBC High | 15 BID po + 0.075 BID ip | |
| 6 | | Vehicle | NA | |
| 7 | NA - Non-EAE controls | | | |

At the onset of the disease (approximately days 10-12) through days 35-40, animals are dosed BID according to the foregoing Table 3. Control groups include DMF alone and two different doses of glibenclamide (Groups 1-3). Dosing is chosen to be well below optimal.. The combinations (Groups 4-5) demonstrate unexpectedly enhanced results over the controls (Groups 1-3) all relative to the vehicle control (Group 6). The vehicle control (Group 6) demonstrates the natural history and magnitude of disease progression in the absence of any intervention. The drug control groups may also receive a matching placebo, e.g., PBS administered IP for the DMF group or 0.8% HPMC administered by oral gavage for the GBC groups, to maintain the blind. Vehicle (PBS and/or 0.8% HPMC) (Goup 6) and non-EAE (Group 7) controls will also be included.

EAE clinical scoring is evaluated visually and is recorded daily beginning on Day 7 until the completion of the study. EAE clinical scores are based on a grading scale of 0 - 5 as described above.

### Example 4 - Enhanced Treatment Effects of Low Dose Dimethyl Fumarate in an NOD Relapsing-Remitting EAE Model in the Presence of Low Dose Glibenclamide

A similar experiment is performerd as in Example 3, except that the study design is as shown in Table 4.

TABLE 4

Study Design NOD/ShiLtJ mice

| Group No. | EAE Induction | Treatment | Dose Level (mg/kg) | Treatment Regimen |
|---|---|---|---|---|
| 1 | MOG + CFA via SC on Days 1 and 8 and two injections of PTx via IP on Days 1 and 2 | DMF | 15 BID po | Daily dosing BID upon onset of disease (~Day 10-12) |
| 2 | | GBC Low | 0.02 BID ip | |
| 3 | | GBC High | 0.075 BID ip | |
| 4 | | DMF + GBC Low | 15 BID po + 0.02 BID ip | |
| 5 | | DMF + GBC High | 15 BID po + 0.075 BID ip | |
| 6 | | Vehicle | NA | |
| 7 | NA- Non-EAE controls | | | |

At the onset of the disease (approximately days 10-12) through days 35-40, animals are dosed BID according to the foregoing Table 4. Control groups include DMF alone and two different doses of glibenclamide (Groups 1-3). Dosing is chosen to be well below optimal.. The combinations (Groups 4-5) demonstrate unexpectedly enhanced results over the controls (Groups 1-3) all relative to the vehicle control (Group 6). The vehicle control (Group 6) demonstrates the natural history and magnitude of disease progression in the absence of any intervention. The drug control groups may also receive a matching placebo, e.g., PBS administered IP for the DMF group or 0.8% HPMC administered by oral gavage for the GBC groups, to maintain the blind. Vehicle (PBS and/or 0.8% HPMC) (Goup 6) and non-EAE (Group 7) controls will also be included.

EAE clinical scoring is evaluated visually and is recorded daily beginning on Day 7 until the completion of the study. EAE clinical scores are based on a grading scale of 0 - 5 as described above.

### Example 5 - Enhanced Treatment Effects of Low Dose Dimethyl Fumarate in an NOD Progressive EAE Model in the Presence of Low Dose Glibenclamide

A similar experiment is performerd as in Example 3, except that the the disease is allowed to transition from relapsing-remitting to progressive. As noted above, unlike the situation in wild-type mice, NOD mice undergo a period of relapsing-remitting through about post-induction day 80-90 and then the disease become progressive. The treatment regimen is detailed in Table 5 below.

TABLE 5

Study Design in NOD/ShiLtJ mice

| Group No. | EAE Induction | Treatment | Dose Level (mg/kg) | Treatment Regimen |
|---|---|---|---|---|
| 1 | MOG + CFA via SC on Days 1 and 8 and two injections of PTx via IP on Days 1 and 2 | DMF | 15 BID po | Daily dosing BID upon onset of progressive disease (~Day 80-90) |
| 2 | | GBC Low | 0.038 BID ip | |
| 3 | | GBC High | 0.075 BID ip | |
| 4 | | DMF + GBC Low | 15 BID po + 0.038 BID ip | |
| 5 | | DMF+GBC High | 15 BID po + 0.075 BID ip | |
| 6 | | Vehicle | NA | |
| 7 | NA- Non-EAE controls | | | |

At the onset of the disease (approximately days 80-90) through days 120-150, animals are dosed BID according to the foregoing Table 5. Control groups include DMF alone and two different doses of glibenclamide (Groups 1-3). Dosing is chosen to be well below optimal.. The combinations (Groups 4-5) demonstrate unexpectedly enhanced results over the controls (Groups 1-3) all relative to the vehicle control (Group 6). The vehicle control (Group 6) demonstrates the natural history and magnitude of disease progression in the absence of any intervention. The drug control groups may also receive a matching placebo, e.g., PBS administered IP for the DMF group or 0.8% HPMC administered by oral gavage for the GBC groups, to maintain the blind. Vehicle (PBS and/or 0.8% HPMC) (Goup 6) and non-EAE (Group 7) controls will also be included.

EAE clinical scoring is evaluated visually and is recorded daily beginning on Day 7 until the completion of the study. EAE clinical scores are based on a grading scale of 0 - 5 as described above.

### Example 6 - Enhanced Treatment Effects of Low Dose Dimethyl Fumarate In An NOD Progressive EAE Model in the Presence of Low Dose Glibenclamide

A similar experiment is performerd as in Example 5, except that the study design is as shown in Table 6.

TABLE 6

Study Design in NOD/ShiLtJ mice

| Group No. | EAE Induction | Treatment | Dose Level (mg/kg) | Treatment Regimen |
|---|---|---|---|---|
| 1 | MOG + CFA via SC on Days 1 and 8 and two injections of PTx via IP on Days 1 and 2 | DMF | 15 BID po | Daily dosing BID upon onset of progressive disease (~Day 80-90) |
| 2 | | GBC Low | 0.02 BID ip | |
| 3 | | GBC High | 0.075 BID ip | |
| 4 | | DMF+GBC Low | 15 BID po + 0.02 BID ip | |
| 5 | | DMF+GBC High | 15 BID po + 0.075 BID ip | |
| 6 | | Vehicle | NA | |
| 7 | NA- Non-EAE controls | | | |

At the onset of the disease (approximately days 80-90) through days 120-150, animals are dosed BID according to the foregoing Table 6. Control groups include DMF alone and two different doses of glibenclamide (Groups 1-3). Dosing is chosen to be well below optimal.. The combinations (Groups 4-5) demonstrate unexpectedly enhanced results over the controls (Groups 1-3) all relative to the vehicle control (Group 6). The vehicle control (Group 6) demonstrates the natural history and magnitude of disease progression in the absence of any intervention. The drug control groups may also receive a matching placebo, e.g., PBS administered IP for the DMF group or 0.8% HPMC administered by oral gavage for the GBC maintain the blind. Vehicle (PBS and/or 0.8% HPMC) (Goup 6) and non-EAE (Group 7) controls will also be included.

EAE clinical scoring is evaluated visually and is recorded daily beginning on Day 7 until the completion of the study. EAE clinical scores are based on a grading scale of 0 - 5 as described above.

### Example 7 -Treatment of Rrms and Spms Patients Using Low Dose Dimethyl Fumarate and Low Dose Glibenclamide

Approximately 450 patients are enrolled aged 18 to 55 years with a diagnosis of RRMS as defined according to the McDonald criteria, a baseline score of 0 to 5.0 on the Expanded Disability Status Scale (EDSS), which ranges from 0 to 10, with higher scores indicating greater disability), and disease activity as evidenced by at least one clinically documented relapse within 12 months before randomization or a brain magnetic resonance imaging (MRI) scan, obtained within 6 weeks before randomization, that showed at least one gadolinium-enhancing lesion. Progressive forms of multiple sclerosis are excluded.

Patients are randomized into three treatment groups: (1) standard of care (approved dose of DMF); (2) half dose of DMF plus low dose glibenclamide; and (3) half dose of DMF plus high dose of glibenclamide. Group 1 receives 120 mg oral DMF twice a day (240 mg per day) for 7 days and then 240 mg twice per day (480 mg per day) for the remainder of the study. Group 2 receives 120 mg oral DMF twice per day (240 mg/day) plus 0.75 mg once per day of oral glibenclamide. Group 3 receives 120 mg DMF twice per day (240 mg/day) plus 0.75 mg of oral glibenclamide twice per day (1.5 mg per day).

Endpoints include annual relapse rate and rate of conversion to secondary progressive disease. Groups 2 and 3, having been administered low dose glibenclamide show surprisingly improved results as compared to Group 1, containing the approved dose of DMF. Improvements include a reduced relapse rate during the relapsing remitting phase of the disease. On longer term follow-up, Groups 2 and 3 show a markedly reduced rate of conversion to progressive disease as compared to Group 1.

What is claimed is:

1. A method of treating multiple sclerosis (MS) in a human patient in need of treatment thereof, the method comprising co-administering to the patient dimethyl fumarate (DMF) or pharmaceutically acceptable salt thereof, and at least one of glibenclamide (GBC), gliclazide (GCZ) or pharmaceutical acceptable salt thereof, wherein the DMF or pharmaceutically acceptable salt thereof is administered at a maintenance dose of equal to or less than about 480 mg/day.

2. The method of claim 1, wherein the DMF is administered at a maintenance dose in an amount of from about 50 mg per day to about 350 mg per day, and wherein the daily maintenance dose of DMF is administered over one, two, three or four doses.

3. The method of claim 2, wherein GBC administered in a dose of between about 0.2 mg per day to about 2 mg per day, or wherein GCZ administered in a dose of between about 1 mg per day to about 60 mg per day.

4. A method of treating multiple sclerosis (MS) in a human patient in need of treatment thereof, the method comprising co-administering to the patient diroximel fumarate (DXF) or pharmaceutically acceptable salt thereof, and at least one of glibenclamide (GBC), gliclazide (GCZ) or pharmaceutical acceptable salt thereof, wherein the DMX or pharmaceutically acceptable salt thereof is administered at a maintenance dose of equal to or less than about 924 mg/day.

5. The method of claim 4, wherein DXF is administered at a maintenance dose in an amount of from about 100 mg per day to about 750 mg per day, and wherein the daily maintenance dose of DMF is administered over one, two, three or four doses.

6. The method of claim 5, wherein GBC administered in a dose of between about 0.2 to about 2 mg per day, or wherein GCZ administered in a dose of between about 1 mg per day to about 60 mg per day.

7. A method of treating multiple sclerosis (MS) in a human patient in need of treatment thereof, the method comprising co-administering to the patient monomethyl fumarate (MMF) or pharmaceutical acceptable salt thereof, and at least one of glibenclamide (GBC), gliclazide (GCZ) or pharmaceutical acceptable salt thereof, wherein MMF is administered at a maintenance dose of equal to or less than about 380 mg/day.

8. The method of claim 7, wherein the MMF is administered at a maintenance dose in an amount of from about 50 mg per day to about 300 mg per day, and wherein the daily maintenance dose of DMF is administered over one, two, three or four doses.

9. The method of claim 8, wherein GBC administered in a dose of between about 0.2 to about 2 mg per day, or wherein GCZ administered in a dose of between about 1 mg per day to about 60 mg per day.

10. The method of claim 1, wherein the DMF, DXF or MMF and the gliclazide or glibenclamide are administered in separate unit dosages.

11. The method of claim 10, wherein the DMF, DXF or MMF and the gliclazide or glibenclamide are administered concurrently.

12. The method of claim 1, wherein the DMF, DXF or MMF and the gliclazide or glibenclamide are administered as a single unit dosage.

13. The method of claim 1, wherein the MS is progressive MS that is selected from the group consisting of primary progressive MS (PPMS), secondary progressive MS (SPMS), progressive-relapsing MS (PRMS) and malignant MS.

14. The method claim 1, wherein the MS is non-progressive relapsing-remitting MS (RRMS).

15. The method of claim 1, wherein the treatment comprises reducing or alleviating one or more symptoms of MS selected from the group consisting of MRI-monitored multiple sclerosis disease activity, relapse rate, accumulation of physical disability, frequency of relapse, frequency of clinical exacerbation, brain atrophy, risk of confirmed progression and time to confirmed disease progression.

16. The method of claim 15, wherein the accumulation of physical disability is measured by the subject’s Kurtzke Expanded Disability Status Scale (EDSS) score.

17. The method of claim 15, wherein the accumulation of physical disability is assessed by the time to confirmed disease progression as measured by Kurtzke Expanded Disability Status Scale (EDSS) score.

18. The method of claim 1, wherein one or more adverse effects of DMF, DXF, MMF or the sulfonylurea are substantially reduced.

19. The method of claim 18, wherein the one or more adverse effects are selected from the group consisting of hypoglycemia, anaphylaxia, angioedema, progressive multifocal leukoencephalopathy, lymphopenia, liver injury, flushing, difficulty with swallowing, dizziness, tachycardia, hives, itching, and skin rash.

20. A method of increasing the effectiveness of dimethyl fumarate when administered to treat a human patient who has multiple sclerosis, comprising administering a first pharmaceutical composition comprising an amount of a fumarate, wherein the amount of the fumarate is administered at a dose lower than its known therapeutically effective dosage amount, and further comprising administering a second pharmaceutical composition comprising an amount of a sulfonylurea, wherein the treatment reduces the incidence of one or more known adverse effects associated with the administration of the fumarate.
